(19) United States
(12) Patent Application Publication
(10) Pub. No.: US 2007/0231332 A1
(43) Pub. Date: Oct. 4, 2007

(54) Inhibition of Cancer Metastasis

(75) Inventors: Behjatolah M. Karbassi, Little Rock, AR (US); Thomas Kieber-Emmons, Little Rock, AR (US)

Correspondence Address: WILLIAMS & ANDERSON PLC, 111 CENTER ST., TWENTY-SECOND FLOOR, LITTLE ROCK, AR, 72201, US

(73) Assignee: Board of Trustees of the University of Arkansas, Little Rock, AR (US)

(21) Appl. No.: 11/694,370

(22) Filed: Mar. 30, 2007

Related U.S. Application Data
(60) Provisional application No. 60/788,018, filed on Mar. 31, 2006.

(51) Int. Cl. A61K 39/395; A61K 38/47; A61K 31/737

(52) U.S. Cl. 424/146.1; 424/94.61; 514/54; 424/178.1

(57) ABSTRACT

P-Selectin on platelets and endothelium binds cell surface chondroitin sulfate (CS) proteoglycans, which are abundantly and stably expressed on the surface many cancer cells. Binding of the cancer cells through the CS moieties may be blocked to inhibit the interaction of cancer cells with platelets and endothelium. The present inventors disclose compositions and methods for the inhibition of cancer metastasis.

### CROSS-REFERENCES TO RELATED APPLICATIONS

This application claims the benefit and priority of U.S. Provisional application No. 60/788,018 filed on Mar. 31, 2006, the contents of which are incorporated herein by reference in their entirety.

### STATEMENT REGARDING FEDERALLY SPONSORED RESEARCH

Development of this invention was supported by grant DAMD17-0101-0366 from the Department of Defense and grant CA089480 from the National Institutes of Health. The United States government may have certain rights in this invention.

### BACKGROUND OF THE INVENTION

Cancer metastasis is strongly correlated with a poor prognosis. The multi-step process of metastasis includes release of malignant cells from the primary neoplasm, migration of cancer cells into circulation, interaction with platelets and leukocytes in circulation, adhesion to endothelium at distant sites, and growth of the disseminated cancer cells within the vessels or within the tissue following extravasation. Each step in this process requires different types of interaction between cancer cells and the host microenvironment.

The selectin family of adhesion molecules plays a significant role in cancer metastases. Selectins may mediate cell to cell interactions between cancer cells and normal cells, including normal cells at a secondary metastatic site. An example of a selectin family member is P-Selectin. P-Selectin is a 140 kDa protein that is commonly expressed on the surface of a variety of cell types, including, but not limited to, platelets and endothelium. (See, for example, GenBank Accession No. P16109 (Homo sapiens) or GenBank Accession No. AAA40008 (Mus musculus).) Another example of a selectin family member is E-Selectin. E-Selectin is commonly expressed in a variety of cell types, including, for example, vascular endothelium. (See, for example, NP_{—}000441 (Homo sapiens) or AAA37577 (Mus musculus).) The oligosaccharides sialyl Lewis X (sLe^{x}) and sialyl Lewis A are known ligands for P- and E-selectin, and these ligands are expressed at elevated levels on various cancer cells of human and murine origin.

Cell surface proteoglycans (PGs) are another class of cell surface adhesion molecules. These PGs may comprise glycosaminoglycan (GAG) side chains covalently bound to a protein core. Heparan sulfate (HS) and chondroitin sulfate (CS) are PGs, and both are P-Selectin ligands.

The mammary cell line 4T1 is a model system of spontaneous breast cancer metastasis. This model exhibits a deficiency in sLe^{x/a} oligosaccharides. This deficiency results in diminished homotypic adhesion and higher motility of the tumor cells. However, while a decrease in homotypic adhesion may help in the release of metastatic cells from the primary tumor, the ability of the circulating tumor cell to extravasate and form secondary metastatic lesions may be disrupted if adhesion to endothelial cells and platelets is also decreased.

### BRIEF SUMMARY OF THE INVENTION

The present inventors demonstrate that P-Selectin binds to chondroitin sulfate proteoglycans on the surface of cancer cells. Additionally, the present inventors demonstrate that platelets which express P-Selectin bind to chondroitin sulfate proteoglycans on the surface of cancer cells through the P-Selectin molecule. The inventors further demonstrate that endothelial cells which express P-Selectin bind to chondroitin sulfate proteoglycans on the surface of cancer cells through the P-Selectin molecule. More importantly, the inventors demonstrate that inhibition of the aforementioned P-Selectin binding to chondroitin sulfate proteoglycans prevents metastasis by preventing tumor cell interaction with platelets or tumor cell interaction with endothelial cells at secondary sites. Inhibition of the interaction of tumor cell chondroitin sulfate proteoglycans with platelets or endothelium may be achieved in multiple ways as set forth herein.

In certain embodiments of the present invention, compositions are disclosed for the inhibition of cancer metastasis. In particular embodiments, such a composition for inhibiting metastasis of a cancer cell may comprise a chondroitin sulfate ligand. In further embodiments, such a composition for inhibiting metastasis of a cancer cell may comprise a P-Selectin ligand. In yet further embodiments, such a composition for inhibiting metastasis of a cancer cell may comprise an inhibitor of synthesis of chondroitin sulfate or sulfation of chondroitin sulfate.

In various embodiments of the present invention, methods of inhibiting metastasis are disclosed. In one embodiment, a method of inhibiting metastasis comprises blocking the interaction of a first cell comprising chondroitin sulfate with a second cell by contacting said first cell with a chondroitin sulfate ligand.

In another embodiment of the present invention, a method of inhibiting metastasis may comprise blocking the interaction of a first cell comprising chondroitin sulfate with a second cell comprising P-Selectin by contacting said second cell with a P-Selectin ligand.

In particular embodiments of the present invention, a method of inhibiting metastasis may comprise contacting a cancer cell with a chondroitin sulfate synthesis inhibitor or a chondroitin sulfate sulfation inhibitor.

### BRIEF DESCRIPTION OF THE DRAWINGS

FIG. 1 illustrates flow cytometry analysis of anti-sialyl Lewis X monoclonal antibody (FH6, KM93, or CSLEX) binding to 4T1 cells transfected with either vector alone (4T1-EGFP) or transfected with vector containing a DNA insert which expresses fucosyl-transferase III (4T1-FTIII). FIG. 1A illustrates analysis of 4T1-EGFP, cells incubated with fluorescein isothiocyanate (FITC) conjugated secondary antibody. FIG. 1B illustrates analysis of 4T1-FTIII cells incubated with FITC-conjugated secondary antibody. FIG. 1C illustrates analysis of 4T1-EGFP cells incubated with FH6 primary antibody followed by FITC-conjugated secondary antibody. FIG. 1D illustrates analysis of 4T1-FTIII cells incubated with FH6 primary antibody followed by FITC-conjugated secondary antibody. FIG. 1E illustrates analysis of 4T1-EGFP cells incubated with KM93 primary antibody followed by FITC-conjugated secondary antibody. FIG. 1F illustrates analysis of 4T1-FTIII cells incubated with KM93 primary antibody followed by FITC-conjugated secondary antibody. FIG. 1G illustrates analysis of 4T1-EGFP cells incubated with CSLEX1 primary antibody followed by FITC-conjugated secondary antibody. FIG. 1H illustrates analysis of 4T1-FTIII cells incubated with CSLEX1 primary antibody followed by FITC-conjugated secondary antibody.

FIG. 2 illustrates E-selectin and P-Selectin binding to 4T1 cells. Either 4T1-EGFP or 4T1-FTIII cells were first incubated with human IgG-chimeric E-selectin or human IgG-chimeric P-Selectin and then stained with FITC-conjugated goat anti-human IgG. 4T1-EGFP cells are illustrated by open histograms and 4T1-FTIII cells are illustrated by filled histograms.

FIG. 3 illustrates calcium dependence of E-selectin and P-Selectin binding to 4T1 cells. FIG. 3A illustrates 4T1 cells stained with human IgG as a control. FIG. 3B illustrates IgG-chimeric E-selectin binding in the absence of EDTA. FIG. 3C illustrates IgG-chimeric E-selectin binding in the presence of 10 mM EDTA. FIG. 3D illustrates IgG-chimeric P-Selectin binding in the absence of EDTA. FIG. 3E illustrates IgG-chimeric P-Selectin binding in the presence of 10 mM EDTA. FIG. 3F illustrates IgG-chimeric P-Selectin binding in the presence of 20 mM EDTA. FIG. 3G illustrates IgG-chimeric P-Selectin binding in the presence of 40 mM EDTA. Mean fluorescence intensity for each histogram is shown.

FIG. 4 illustrates the effect of neuraminadase treatment of 4T1 cells on P-Selectin binding. Filled histogram represents staining with secondary antibody only. Continuous line histogram represents P-Selectin reactivity without neuraminidase treatment. Dotted histogram represents P-Selectin reactivity with neuraminidase treatment.

FIG. 5 illustrates that pronase treatment of 4T1 cells reduces P-Selectin reactivity with the cells. P-Selectin binding to the 4T1 cells (thin line histogram) was sharply reduced (dotted line) to the level of secondary antibody binding (thick solid line).

FIG. 6 illustrates that inhibition of sulfation decreases P-Selectin binding to 4T1 cells. FIG. 6A illustrates P-Selectin binding to untreated cells. FIG. 6B illustrates P-Selectin binding to cells treated to inhibit sulfation.

FIG. 7 illustrates the effect of heparinase and chondroitinase on P-Selectin binding. FIG. 7A illustrates untreated cells. FIG. 7B illustrates P-Selectin binding to untreated cells. FIG. 7C illustrates P-Selectin binding to cells treated with heparinase and chondroitinase.

FIG. 8 illustrates histochemical binding of P-Selectin to the primary mass and metastatic pulmonary tumors. P-Selectin ligands were expressed uniformly and strongly on cells of the primary mass in both parental 4T1 cells (FIG. 8A) and sialyl-Lewis X Negative cells (sLe^{x}-Neg) (FIG. 8B). P-Selectin ligands were very strongly expressed on metastatic cells in lung sections (FIG. 8C). Bar equals 20 μm.

FIG. 9 illustrates involvement of P-Selectin ligands in binding to human vascular endothelial cells. Percentage of adhesion was calculated based on mean fluorescence intensities and presented as average of 11 replications. Bars represent SD based on 11 replications. A representative experiment out of three is shown. Paired Student's t test was used to compare the means.

FIG. 10 illustrates the effect of heparin on P-Selectin interaction with sLe^{x}-Neg tumor cells. FIG. 10A illustrates the tumor cells incubated with secondary antibody alone. FIG. 10B illustrates the interaction of P-Selectin with the tumor cells when the P-Selectin had been pre-incubated with 0.7 Units heparin prior to exposure to the cells. FIG. 10C illustrates the interaction of P-Selectin with the tumor cells when the P-Selectin had been pre-incubated with 3.0 Units heparin prior to exposure to the cells. FIG. 10D illustrates the interaction of P-Selectin with the tumor cells when the P-Selectin had been pre-incubated with 15.0 Units heparin prior to exposure to the cells. FIG. 10E illustrates the interaction of P-Selectin with the tumor cells when the P-Selectin had been pre-incubated with 60.0 Units heparin prior to exposure to the cells. FIG. 10F illustrates the interaction of P-Selectin with the tumor cells when the P-Selectin had been pre-incubated with 120.0 Units heparin prior to exposure to the cells.

FIG. 11 illustrates that heparin inhibits binding of mouse platelets to 4T1 cells. FIG. 11A illustrates lack of binding of 4T1 cells incubated with untreated platelets. FIG. 11B illustrates binding of 4T1 cells to platelets which had been pre-treated with thrombin. FIG. 11C illustrates that heparin can inhibit binding of 4T1 cells to platelets which had been pre-treated with thrombin.

FIG. 12 illustrates that chondroitin sulfates are P-Selectin ligands on 4T1 cells. FIG. 12A illustrates 4T1 cells incubated with secondary antibody alone. FIG. 12B illustrates P-Selectin binding to 4T1 cells. FIG. 12C illustrates P-Selectin binding to 4T1 cells which had been pre-treated with heparinase. FIG. 12D illustrates P-Selectin binding to 4T1 cells which had been pre-treated with chondroitinase. FIG. 12E illustrates P-Selectin binding to 4T1 cells which had been pre-treated with both heparinase and chondroitinase.

FIG. 13 illustrates inhibition of P-Selectin binding to 4T1 cells by chondroitin sulfate. FIG. 13A illustrates 4T1 cells incubated with secondary antibody alone. FIG. 13B illustrates P-Selectin binding to 4T1 cells. FIG. 13C illustrates P-Selectin binding to 4T1 cells when the P-Selectin had been pre-treated with 5.0 mg/ml chondroitin sulfate A. FIG. 13D illustrates P-Selectin binding to 4T1 cells when the P-Selectin had been pre-treated with 5.0 mg/ml chondroitin sulfate B. FIG. 13E illustrates P-Selectin binding to 4T1 cells when the P-Selectin had been pre-treated with 0.5 mg/ml chondroitin sulfate A. FIG. 13F illustrates P-Selectin binding to 4T1 cells when the P-Selectin had been pre-treated with 0.5 mg/ml chondroitin sulfate B. FIG. 13G illustrates P-Selectin binding to 4T1 cells when the P-Selectin had been pre-treated with 0.05 mg/ml chondroitin sulfate A. FIG. 13H illustrates P-Selectin binding to 4T1 cells when the P-Selectin had been pre-treated with 0.05 mg/ml chondroitin sulfate B. FIG. 13I illustrates P-Selectin binding to 4T1 cells when the P-Selectin had been pre-treated with 0.005 mg/ml chondroitin sulfate A. FIG. 13J illustrates P-Selectin binding to 4T1 cells when the P-Selectin had been pre-treated with 0.005 mg/ml chondroitin sulfate B.

FIG. 14 illustrates inhibition of P-Selectin binding to 4T1 cells by chondroitin sulfate. FIG. 14A illustrates 4T1 cells incubated with secondary antibody alone. FIG. 14B illustrates P-Selectin binding to 4T1 cells. FIG. 14C illustrates P-Selectin binding to 4T1 cells when the P-Selectin had been pre-treated with 0.5 mg/ml chondroitin sulfate E. FIG. 14D illustrates P-Selectin binding to 4T1 cells when the P-Selectin had been pre-treated with 0.05 mg/ml chondroitin sulfate E. FIG. 14E illustrates P-Selectin binding to 4T1 cells when the P-Selectin had been pre-treated with 0.005 mg/ml chondroitin sulfate E. FIG. 14F illustrates P-Selectin binding to 4T1 cells when the P-Selectin had been pre-treated with 0.0005 mg/ml chondroitin sulfate E.

FIG. 15 illustrates that CS PGs are the main P-Selectin ligands on the surface of a human breast cancer cell line. Inhibition of P-Selectin binding to metastatic human breast cancer cells chondroitinase or a mixture of glycosaminoglycans and chondroitin sulfate is illustrated. FIG. 15A illustrates MDA-MET cell variant, which is a bone-colonizing variant of MDA-MB-231 cell line, incubated with secondary antibody alone. FIG. 15B illustrates P-Selectin binding to MDA-MET cells. FIG. 15C illustrates P-Selectin binding to MDA-MET cells when the cells had been pre-treated with heparinase. FIG. 15D illustrates P-Selectin binding to MDA-MET cells when the cells had been pre-treated with chondroitinase. FIG. 15E illustrates P-Selectin binding to MDA-MET cells when the cells had been pre-treated with both heparinase and chondroitinase. FIG. 15F illustrates P-Selectin binding to MDA-MET cells when the P-Selectin had been pre-treated with 10.0 mg/ml of a chondroitin sulfate and glycosaminoglycan mixture. FIG. 15G illustrates P-Selectin binding to MDA-MET cells when the P-Selectin had been pre-treated with 1.0 mg/ml of a chondroitin sulfate and glycosaminoglycan mixture.

### DETAILED DESCRIPTION

In certain embodiments of the present invention, compositions are disclosed for the inhibition of cancer metastasis. In particular embodiments, such a composition for inhibiting metastasis of a cancer cell may comprise a chondroitin sulfate ligand. In other embodiments, a composition for inhibiting metastasis of a cancer cell may comprise a P-Selectin ligand.

CS proteoglycans on the surface of cancer cells are shown to be major P-Selectin ligands involved in prometastatic heterotypic adhesion of tumor cells to platelets or endothelial cells. Metastasis may be inhibited by contacting platelets or endothelial cells with a P-Selectin ligand thereby preventing the interaction of platelets or endothelial cells with cancer cells. Thus, one aspect of the present invention provides for a metastasis inhibiting composition comprising a P-Selectin ligand that blocks the binding of P-Selectin to chondroitin sulfate on cancer cells. Such a P-Selectin ligand may be, for example, chondroitin sulfate.

One aspect of the present invention provides for a metastasis inhibiting composition comprising chondroitin sulfate. Yet a further aspect of the present invention provides for a metastasis inhibiting composition comprising a chondroitin sulfate binding agent which blocks the binding of P-Selectin to chondroitin sulfate. The particular CS that may be useful according to the present embodiment may be any of a variety of CS molecules including, but not limited to, CS PGs, CS A, CS B, CS C, CS D, or CS E. Additionally, inhibition of binding of platelets comprising P-Selectin to cancer cells which comprise cell surface CS PGs can be achieved by contacting the P-Selectin on platelets with free or unbound CS thereby inhibiting metastasis. Similarly, binding of endothelial cells comprising P-Selectin to cancer cells which comprise cell surface CS PGs can be blocked by contacting the P-Selectin on endothelial cells with free or unbound CS thereby inhibiting metastasis. The free or unbound chondroitin sulfate may be free or unbound CS PGs, CS A, CS B, CS C, CS D, or CS E.

In a further aspect of the present invention, binding of P-Selectin to CS PGs on the surface of cancer cells can be prevented by contacting the CS on cancer cells with a chondroitin sulfate ligand or binding agent. Free or unbound P-Selectin or a chondroitin sulfate binding domain of P-Selectin may be contacted to the cancer cell. In this manner, the free P-Selectin or chondroitin sulfate binding domain of P-Selectin may bind to the chondroitin sulfate of the cancer cells and prevent the interaction of cancer cells with cells comprising P-Selectin, such as platelets or endothelium. Because the chondroitin sulfate of the cancer cells is bound, metastasis is inhibited.

The extent of synthesis of chondroitin sulfate and sulfation of chondroitin sulfate is relevant to the binding of chondroitin sulfate to P-Selectin. Therefore, in certain embodiments of the present invention, a composition for inhibiting metastasis of a cancer cell may comprise an inhibitor of synthesis chondroitin sulfate. Such an inhibitor would include an inhibitor of sulfation of chondroitin sulfate. By decreasing the synthesis or sulfation of chondroitin sulfate on tumor cells, the binding of chondroitin sulfate by P-Selectin is limited. As a result, it is possible to limit the metastasis of a cancer cell by inhibiting sulfation of chondroitin sulfate.

In various embodiments of the present invention, methods of inhibiting metastasis are disclosed. In one embodiment, a method of inhibiting metastasis comprises blocking the interaction of a first cell comprising chondroitin sulfate with a second cell by contacting said first cell with a chondroitin sulfate ligand.

In another embodiment of the present invention, a method of inhibiting metastasis may comprise blocking the interaction of a first cell comprising chondroitin sulfate with a second cell comprising P-Selectin by contacting said second cell with a P-Selectin ligand.

In various aspects of the present invention, methods are disclosed to inhibit metastasis by inhibiting the interaction of P-Selectin expressed on platelets or endothelium with chondroitin sulfate proteoglycans expressed on tumor cells.

In various aspects of the present invention, methods are disclosed to inhibit metastasis by inhibiting the interaction of P-Selectin expressed on endothelial cells with chondroitin sulfate proteoglycans expressed on tumor cells.

In yet another aspect of the present invention, binding of P-Selectin on platelets or P-Selectin on endothelial cells to CS PGs on the surface of cancer cells can be prevented by contacting the CS PGs on the surface of cancer cells with a chondroitin sulfate binding agent that inhibits or blocks the P-Selectin binding site. Such a chondroitin sulfate ligand or binding agent would be, such as, for example, free P-Selectin or such as, for example, anti-CS antibodies.

In a further aspect of the present invention, CS can be utilized to stimulate an immune response, thereby inducing CS-specific antibodies that block the interaction of P-Selectin with CS bound to tumor cells. Antibodies for such a strategy may be generated in vivo or in vitro. Such antibodies inhibit metastasis via active immunization or passive immunization.

In particular embodiments of the present invention, a method of inhibiting metastasis may comprise contacting a cancer cell with a chondroitin sulfate synthesis or sulfation inhibitor. It is within the scope of the present invention that disruption of the enzymatic pathways that result in CS production or other cellular pathways that result in P-Selectin production may be useful for inhibiting the interaction of CS of tumor cells with P-Selectin of platelets or P-Selectin of endothelial cells thereby inhibiting metastasis. In particular, a method of inhibiting metastasis may comprise contacting a cancer cell with a chondroitin sulfate synthesis or chondroitin sulfate sulfation inhibitor. Such an inhibitor may inhibit sulfation of chondroitin sulfate, thereby inhibiting the effectiveness of P-Selectin binding to chondroitin sulfate. As a result, metastasis is inhibited. Exemplary inhibitors include inhibitors of cellular enzymes that are involved in the synthesis of chondroitin sulfate. Particular enzymes include, but are not limited to, chondroitin synthase, chondroitin N-acetylgalactosaminyltransferase (Chondroitin GalNAcT), chondroitin-glucuronate C5-epimerase, chondroitin 4-O-sulfotransferase-1 (C4ST1), chondroitin 4-O-sulfotransferase-2 (C4ST2), chondroitin 4-O-sulfotransferase-3 (C4ST3), dermatan 4-O-sulfotransferase-1 (D4ST1), chondroitin 6-O-sulfotransferase (C6ST), chondroitin 6-O-sulfotransferase-2 (C6ST2), chondroitin 4-sulfate 6-O-sulfotransferase (GalNAc4S-6ST) and galactosaminyl uronyl 2-O sulfotransferase (CS/DS2ST). Inhibition of any of these enzymes may be achieved by any of a variety of compositions and methods. For example, small molecule inhibitors of an enzyme may be used. Alternatively, the expression of particular enzymes may be down-regulated through molecular biology techniques that are commonly known to one of skill in the relevant art. For example, anti-sense RNAs from anti-sense constructs or siRNA (short interfering RNAs) may be used to disrupt translation and thereby inhibit expression.

It is within the scope of various aspects of this invention that metastasis may be inhibited for numerous cancers including, but not limited to, cancers selected from the group consisting of colon cancer, lung cancer, breast cancer, malignant melanoma, gastric cancer, tongue squamous cancer, myeloma and neuroblastoma.

In various aspects of the present invention, methods are disclosed to inhibit metastasis by inhibiting the interaction of P-Selectin expressed on platelets with chondroitin sulfate proteoglycans expressed on tumor cells. In various aspects of the present invention, methods are disclosed to inhibit metastasis by inhibiting the interaction of P-Selectin expressed on endothelial cells with chondroitin sulfate proteoglycans expressed on tumor cells.

In one aspect of the present invention, CS proteoglycans on the surface of cancer cells are shown to be major P-Selectin ligands involved in prometastatic heterotypic adhesion of tumor cells to platelets or endothelial cells. In another aspect of the present invention, metastasis may be inhibited by contacting platelets or endothelial cells with a P-Selectin ligand thereby preventing the interaction of platelets or endothelial cells with cancer cells.

In a further aspect of the present invention, binding of P-Selectin to CS PGs on the surface of cancer cells can be prevented by contacting the P-Selectin with free or unbound CS thereby inhibiting metastasis. Additionally, inhibition of binding of platelets or endothelium which comprise P-Selectin to cancer cells which comprise cell surface CS PGs can be achieved by contacting the P-Selectin on platelets with free or unbound CS. The free or unbound chondroitin sulfate may be free or unbound CS PGs, CS A, CS B, CS C, CS D, CS E.

In yet a further aspect of the present invention, binding of P-Selectin to CS PGs on the surface of cancer cells can be prevented by contacting the P-Selectin with a P-Selectin ligand, such as a small molecule, that prevents, blocks or inhibits binding of P-Selectin to CS thereby inhibiting metastasis. Additionally, inhibition of binding of platelets which comprise P-Selectin to cancer cells which comprise cell surface CS PGs can be achieved by contacting the P-Selectin on platelets with a P-Selectin ligand that prevents, blocks or inhibits binding of P-Selectin to CS, thereby inhibiting metastasis. Similarly, binding of endothelial cells which comprise P-Selectin to cancer cells which comprise cell surface CS PGs can be achieved by contacting the P-Selectin on endothelial cells with a P-Selectin ligand that prevents, blocks or inhibits binding of P-Selectin to CS thereby inhibiting metastasis. It is also envisioned within the scope of the present invention that modified forms of CS chains with improved specificity for P-Selectin or peptides that mimic the clustering structure of tumor cell surface CS may also be used for inhibition of metastasis.

It is within the scope of the various compositions and methods of this invention that metastasis may be inhibited for numerous cancers including, but not limited to, cancers selected from the group consisting of colon cancer, lung cancer, breast cancer, malignant melanoma, gastric cancer, tongue squamous cancer, myeloma and neuroblastoma.

### EXAMPLES

The following examples are further illustrative of the present invention, but it is understood that the invention is not limited thereto.

The monoclonal antibody KM-93 was purchased from Kamiya Biomedical, Seattle, Wash. The antibodies FH6 and CSLEX1 were purchased from GlycoTech, Gaithersburg, Md. FITC-conjugated and biotinylated goat anti-mouse IgG or goat anti-mouse IgM were purchased from Sigma.

The murine breast tumor cell line 4T1 was obtained from ATCC (Manassas, Va.). The 4T1 cell line, FTIII transfected 4T1 cell line and pIRES-EGFP transfected cell line were maintained in DMEM supplemented with 10% fetal bovine serum at 37° C. in sterile culture flasks.

The 1083 bp coding fragment of the human fucosyl transferase III (FTIII) gene (see GenBank Accession Nos. NP_{—}000140 and U27328.1) in pCDNA3 plasmid was kindly provided by Dr. Insug O'Sullivan (University of Illinois). The coding sequence was further adapted for cloning between EcoRI and XhoI restriction sites by PCR using the following primers: 5′-cgagaattctcaggtgaaccaagccgctatg-3′ (SEQ ID NO.: 1) and 5′-cgactcgagatggatcccctgggtgca-3′ (SEQ ID NO.: 2). The amplified fragment was digested with EcoRI and XhoI, purified and inserted into the Multiple Cloning Site (MCS) of pIRES-EGFP vector to make FTIII-pIRES-EGFP construct. (The pIRES-EGFP vector was obtained from BD Biosciences Clontech (Palo Alto, Calif.).) The 4T1 cells were then transfected with this construct or pIRES-EGFP vector alone using Lipofectamine™ 2000 (Invitrogen, Carlsbad, Calif.) transfection reagent. The pIRES-EGFP vector contains an internal ribosome entry site (IRES) between the MCS and the EGFP (enhanced green fluorescent protein) coding region. This allows the FTIII gene (cloned into the MCS) and the EGFP gene to be translated from a single bicistronic mRNA.

Unless specified otherwise, flow cytometry was conducted as follows. Acquisition and analysis of data was performed on an EPICS®XL™ flow cytometer (Beckman Coulter, Inc., Fullerton, Calif.). Cells were passed to new flasks 24 hours before measuring lectin binding. The subconfluent monolayer of cells was detached with Cellstripper (Mediatech, Inc. Herndon, Va.) and washed with Dulbecco's phosphate buffered saline with Ca++ and Mg++ (Mediatech, Inc. Herndon, Va.). Cells were transferred to FACS buffer (Dulbecco's Phosphate Buffered Saline, 1% BSA and 0.1% Sodium Azide), counted and diluted to ˜1-2×10⁶/ml. Monoclonal antibodies were added to a final concentration of 10 μg/ml. Cells were incubated on ice for 30 minutes, washed twice with FACS buffer, before the addition of FITC-conjugated streptavidin (2 μg/ml) for lectin analysis or FITC-conjugated goat anti-mouse immunoglobulin for monoclonal analysis. Cells were then washed and fixed with paraformaldehyde, before analysis by flow cytometry.

Recombinant E- and P-Selectin/Fc (human IgG) were purchased from R&D systems, Minneapolis, Minn. These recombinant molecules and FITC-conjugated anti-human IgG were used for binding analyses in flow cytometry assays. Human and murine recombinant selectins were used for human and murine cells, respectively.

All experiments were repeated at least three times. The Student's t-test or Fisher exact test was used to compare differences between means. Differences were considered significant if P was <0.05.

### Example 1

4T1 cells are deficient in sLe^{a} expression making the cell line a good candidate to study the involvement of sLe^{x}-mediated adhesion properties. There are several monoclonal antibodies (mAbs) defined as KM93, FH6 and CSLEX1 that recognize sLe^{x}. These mAbs recognize different forms of the sLe^{x} antigen (1-3). FH6 is specific for an extended form of sLe^{x} (4), while CSLEX1 and KM93 antibodies both recognize the sLe^{x} tetrasaccharide. However, the nature of the molecules carrying the carbohydrate determinant is known to affect the reactivity of CSLEX1 and KM93 (5). Among the above antibodies, only KM93 reacts with the 4T1 tumor cell surface. KM93, CSLEX-1 or FH6 reactive sLe^{x} epitopes may differentially react with P- and E-selectin due to variations in lipid or peptide backbones.

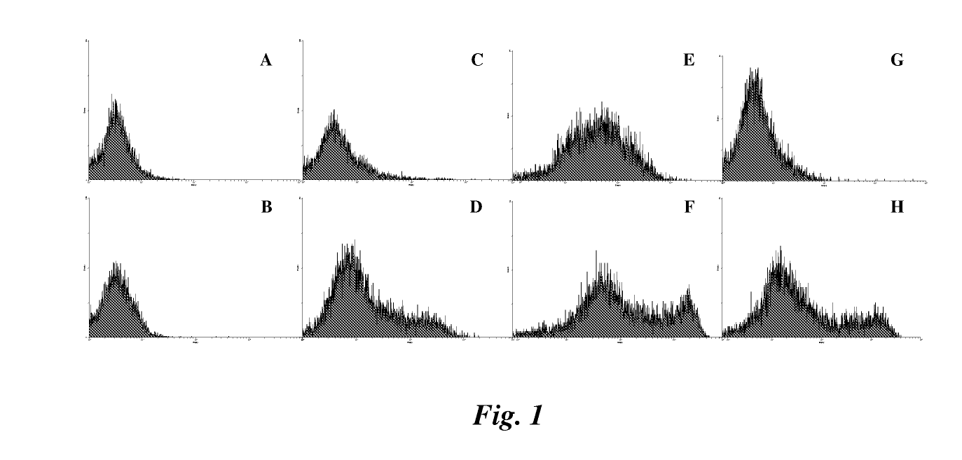

The 4T1 cells were transfected with fucosyltransferase III (FTIII) to expand the expression of other sLe^{x} epitopes (4T1-FTIII). The 4T1 cells were also transfected with pIRES-EGFP vector alone as a control (4T1-EGFP). FIGS. 1A and 1B illustrate 4T1-EGFP cells and 4T1-FTIII cells, respectively, incubated with secondary antibody alone as control. The binding of KM93 monoclonal antibody was increased on 4T1-FTIII cells (FIG. 1F) relative to 4T1-EGFP (FIG. 1E). More importantly, FH6 reactive epitopes were expressed at detectable levels in the 4T1-FTIII cells (FIG. 1D) compared to 4T1-EGFP cells (FIG. 1C). Also, CSLEX-1 reactive epitopes were expressed at detectable levels in the 4T1-FTIII cells (FIG. 1H) compared to 4T1-EGFP cells (FIG. 1G). The antibody binding data indicate that transfection with FTIII encoding sequence increased the expression of various sLe^{x} epitopes.

### Example 2

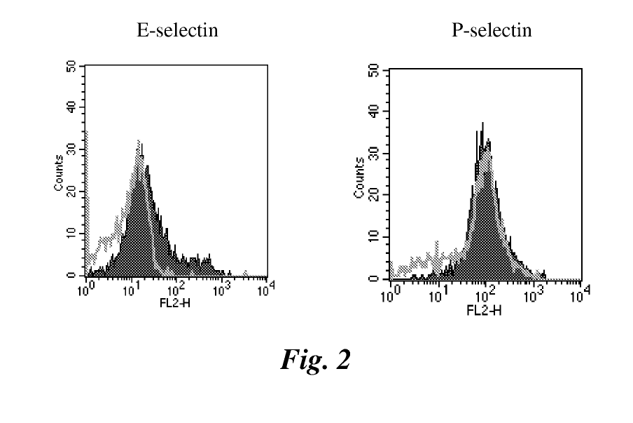

P-Selectin and E-Selectin reactivity with parental and transfected 4T1 cells were examined. Cells were incubated with recombinant mouse E-selectin/Fc (human IgG) or P-Selectin/Fc (human IgG) chimeras and assayed for binding by flow cytometry. An increase for E-selectin binding was observed after FT-III transfection (FIG. 2, first panel). This was expected given the increase in KM93, CSLEX-1 and FH6 binding after transfection. P-Selectin, however, bound very well to the parental 4T1 cells and the binding did not increase for the transfected cells (FIG. 2, second panel). These data confirm that P-Selectin binding to 4T1 cells is not dependent on the expression sLe^{x} on the tumor cell surface.

### Example 3

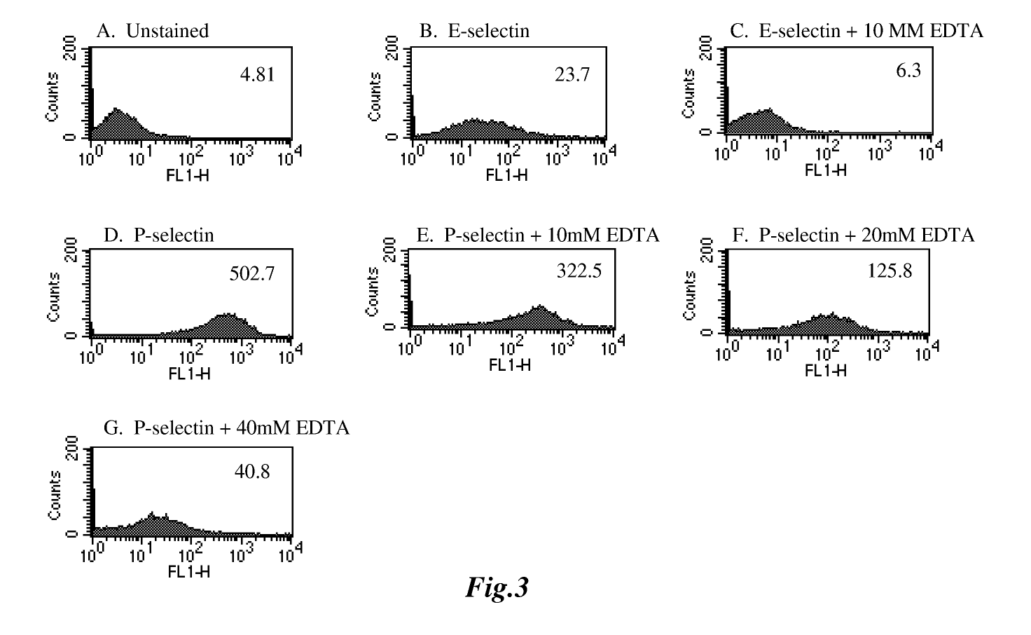

The dependence of E-selectin and P-Selectin binding to 4T1 cells on divalent cation concentration was examined. FIG. 3A illustrates unstained 4T1 cells. FIG. 3B illustrates 4T1 cells incubated with E-selectin/Fc chimera followed by incubation with FITC-conjugated anti-human IgG. FIG. 3C is the same as FIG. 3B except the experiment was conducted in the presence of 10 mM EDTA. Both lectins bind the 4T1 cells, with P-Selectin showing very strong reactivity (FIG. 3D). Contrary to the E-selectin reactivity (see FIGS. 3B and 3C), P-Selectin reactivity was not blocked by a low concentration of EDTA (see FIGS. 3D and 3E). EDTA inhibited P-Selectin reactivity only at high concentrations indicating the Ca⁺⁺-independent nature of the reactivity (see FIGS. 3F and 3G).

### Example 4

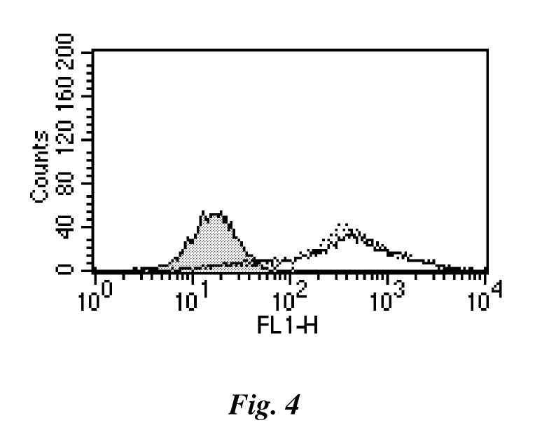

Cells treated with neuraminidase show the relationship between sialylation and reactivity of P-Selectin. Neuraminidase (Vibrio cholerae) was purchased from Sigma (St. Louis, Mo.) and used at a concentration of 50 mU/ml. Neuraminidase treatment did not change the P-Selectin reactivity (FIG. 4). These results provide further evidence that E-selectin and P-Selectin react with separate ligands on the surface of 4T1 cells. In contrast to E-selectin, P-Selectin binds to unsialylated ligands on these cells in a Ca²⁺-independent manner. Therefore, sialylated ligands are not a major ligand of P-Selectin in binding to the 4T1 cells.

### Example 5

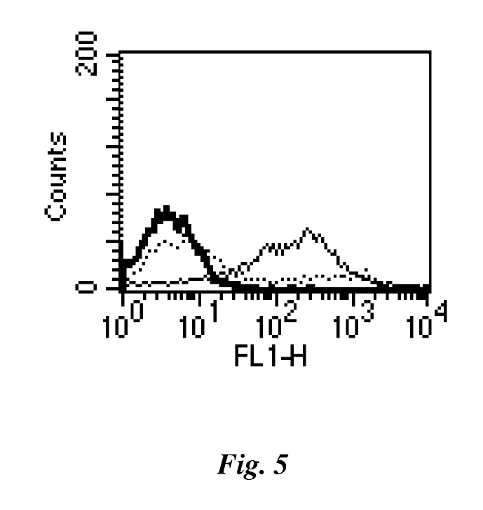

Cells (4T1) were treated with pronase to determine the proteinaceous nature of P-Selectin ligands. Treatment with pronase dropped the P-Selectin reactivity (dotted histogram) almost to the levels of the negative control (thick, solid line histogram), indicating the proteinaceous nature of the ligands (FIG. 5). The thin, solid line histogram represents P-Selectin binding to untreated cells.

### Example 6

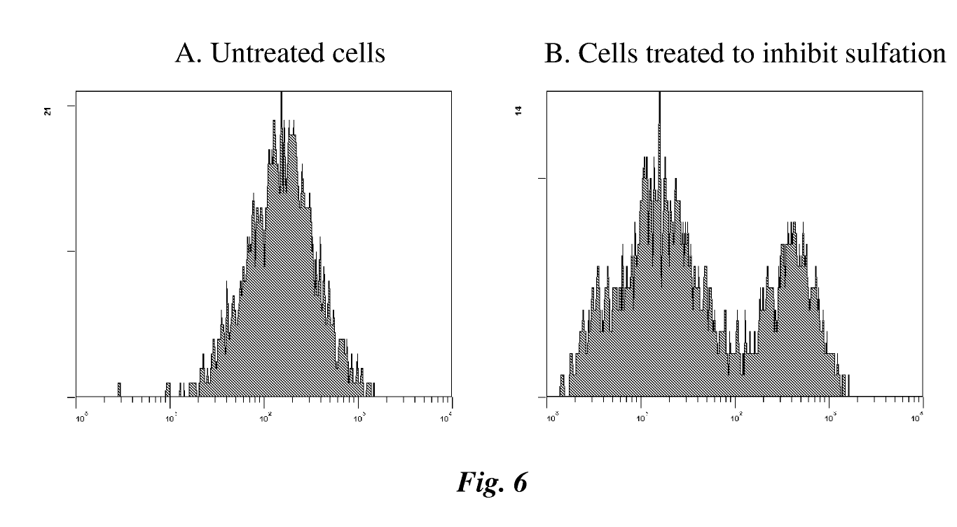

Sulfated glycosaminoglycans like heparan sulfate and chondroitin sulfate are carbohydrate moieties of proteoglycans, which serve as P-Selectin ligands (6, 7). The 4T1 cells were grown in sulfate-free medium in the presence of sodium chlorate to inhibit sulfate biosynthesis. Cells were washed with sulfate-free DMEM medium (Hyclone, Logan, Utah) supplemented with 10% dialyzed FBS and 100 mM sodium chlorate (Sigma) and cultured in the same medium for 2 hours. The medium was then refreshed and incubation was continued overnight. These treated cells were harvested with cell dissociation buffer (Gibco-Invitrogen. Carlsbad, Calif.), washed and resuspended in FACS buffer for further analyses by flow cytometry. Growing the cells in sulfate free medium containing sodium chlorate led to elimination of P-Selectin binding in a majority of the cells, indicating that most P-Selectin ligands on the 4T1 cells are sulfated (FIG. 6).

### Example 7

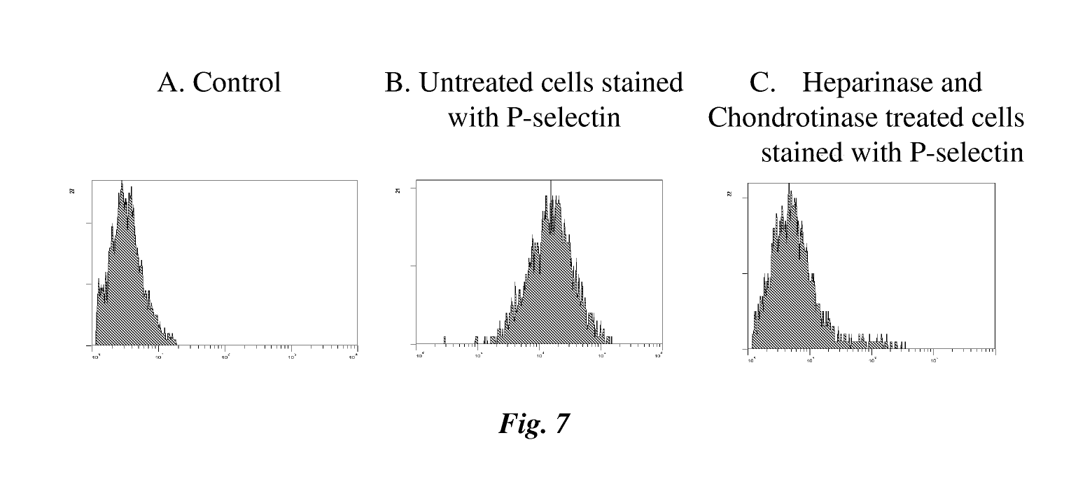

Treatment of 4T1 cells with a mixture of the glycosaminoglycan-cleaving enzymes, heparinase and chondroitinase, decreased P-Selectin binding (FIG. 7). Removal of glycosaminoglycans was performed by treatment of 2×10⁵ cells with a mixture of heparinase II (25 units/ml, Sigma, St. Louis, Mo.) and chondroitinase ABC (5 units/ml, Sigma) in 500 μl of HBSS buffer for 1 hour at 37° C. Alternatively, the above preparation was treated with 500 μg pronase (EMD Biosciences, San Diego, Calif.) for 45 minutes at 37° C. Removal of sialic acid was performed by incubating cells with 50 mU/ml neuraminidase from Vibrio cholerae (Sigma) at 37° C. for 1 hour. These data indicate that P-Selectin ligands on the surface of 4T1 cells are sulfated proteoglycans, most likely the glycosaminoglycans heparan sulfate or chondroitin sulfate.

### Example 8

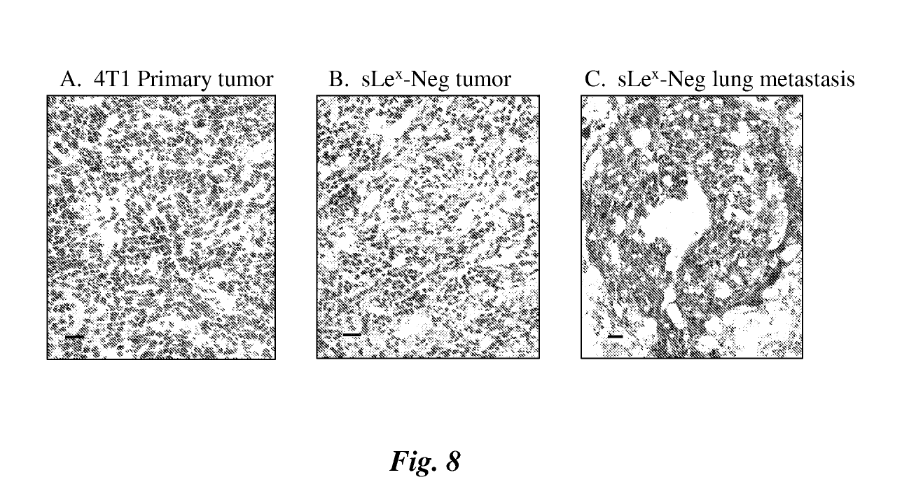

P-Selectin ligands are stably expressed on the surface of 4T1 cells. To examine the stability of expression in vivo, pathological samples from primary 4T1 and 4T1 sLe^{x}-Neg variant tumors were stained (FIG. 8). P-Selectin histochemistry was performed as follows: primary tumors and lungs were harvested from mice inoculated with the 4T1 cells at 21 days post inoculation, placed in optimal cutting temperature compound (Ted Pella Inc., Redding, Calif.) and frozen in liquid nitrogen. Five micron frozen sections were fixed for 10 minutes in cold acetone and then washed with cold DPBS (Cellgro® Mediatech, Herndon, Va.). Endogenous peroxidase was blocked by immersion in 0.3% (w/v) hydrogen peroxide in absolute methanol for 15 minutes followed by DPBS wash. Non-specific binding was blocked by incubating with DPBS+1% BSA at room temperature for 20 minutes. Sections were then incubated with recombinant mouse P-Selectin/human FC chimera (R&D systems, Minneapolis, Minn.) for 30 minutes in DPBS+0.2% BSA at room temperature and then washed in DPBS. Sections were incubated with anti-human IgG (Fc specific) peroxidase conjugate (1/300 dilution) for 15 minutes at room temperature followed by DPBS wash. Sections were incubated with diaminobenzidine solution (DAB) for 5 minutes at room temperature, washed with distilled water, counterstained with methyl green, mounted, and examined under a light microscope. Primary antibody was omitted in negative controls to rule out non-specific binding of the secondary antibody. P-Selectin ligands were observed to be significantly and stably expressed on the surface of tumor cells in the primary and secondary lesions, and expression was similar for both 4T1 and sLe^{x/a}-Neg variant tumors. Therefore, P-Selectin ligands play a role in hematogenous metastasis in this syngeneic breast cancer model.

### Example 9

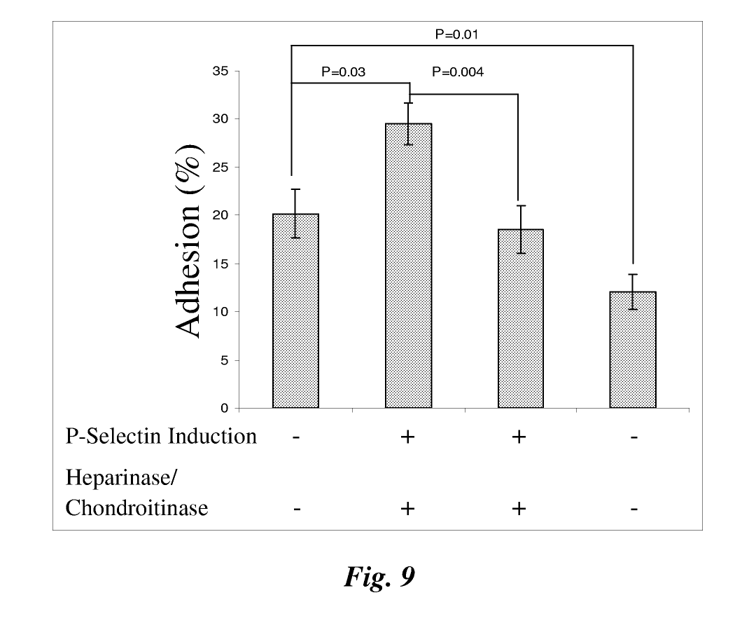

Interaction of P-Selectin and its ligands play an important role in 4T1 cells binding to HUVECs (FIG. 9). To measure tumor cell adhesion to endothelial cells, Clonetics™ human umbilical vein endothelial cell (HUVEC) system was used (Cambrex Biosciences, Walkersville, Md.). A monolayer of HUVEC cells was prepared. HUVECs were incubated with supplied medium supplemented with IL-4 (20 ng/ml) for 24 hours. Medium was replaced with similar medium supplemented with Prostaglandin E2 (PGE2) for 10 minutes. Calcein AM-labeled (Molecular Probes, Eugene, Oreg.) 4T1 cells were treated with chondroitinase/heparinase, then added to the activated monolayers of HUVECs. Cells were co-incubated at 37° C. for 30 minutes and then unbound 4T1 cells were removed by washing gently with pre-warmed medium. PBS was added to all wells and fluorescence measured and percentage of adhesion was calculated. Stimulating surface expression of P-Selectin on HUVECs led to an increase in adhesion to the 4T1 cells. The adhesion to 4T1 cells was significantly inhibited by treatment with the mixture of heparinase and chondroitinase. There was background adhesion to HUVECs, which was also significantly inhibited by treating the 4T1 cells with heparinase/chondroitinase mix, implying a constitutive presence of P-Selectin on the HUVECs under our experimental conditions. This was confirmed by examining P-Selectin expression on HUVECs. A low constitutive expression of P-Selectin was detected on 10% of cells, which was elevated to a more intense staining on about 20% of cells after treatment with IL-4 and PGE2. Adhesion was clearly enhanced after P-Selectin induction on HUVECs and suppressed after heparinase/chondroitinase treatment of tumor cells (FIG. 9).

### Example 10

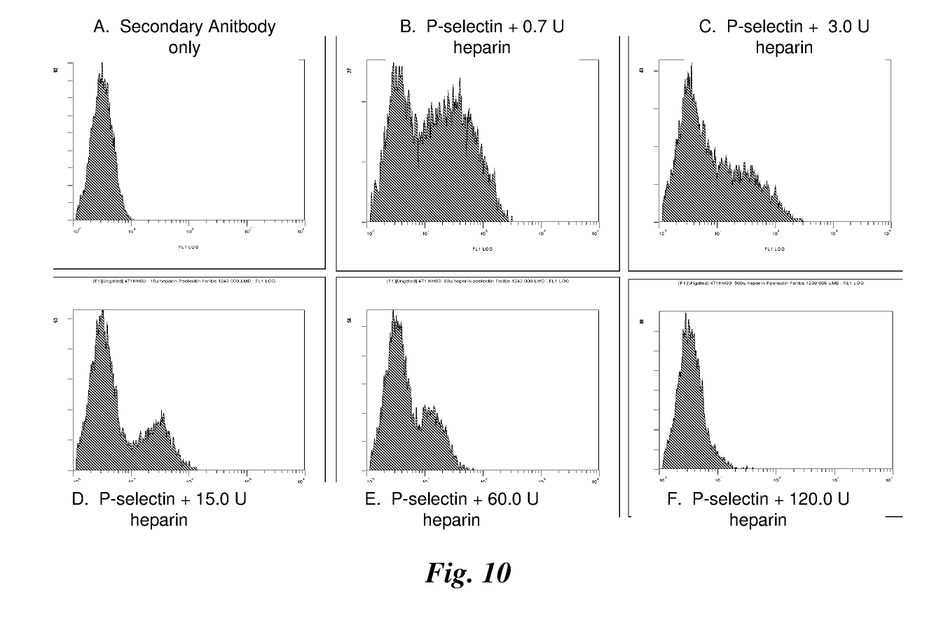

Heparin inhibits both P-Selectin binding to the tumor cells and tumor cell-platelet interactions mediated by P-Selectin. Heparin's ability to inhibit P-Selectin interaction with the cell surface in vitro was tested using the sLe^{x}-Neg 4T1 cell variant. Recombinant P-Selectin was incubated with heparin and then the mixture was added to cells to test the binding (FIG. 10). Heparin efficiently inhibited P-Selectin binding to cells in a dose dependent manner.

### Example 11

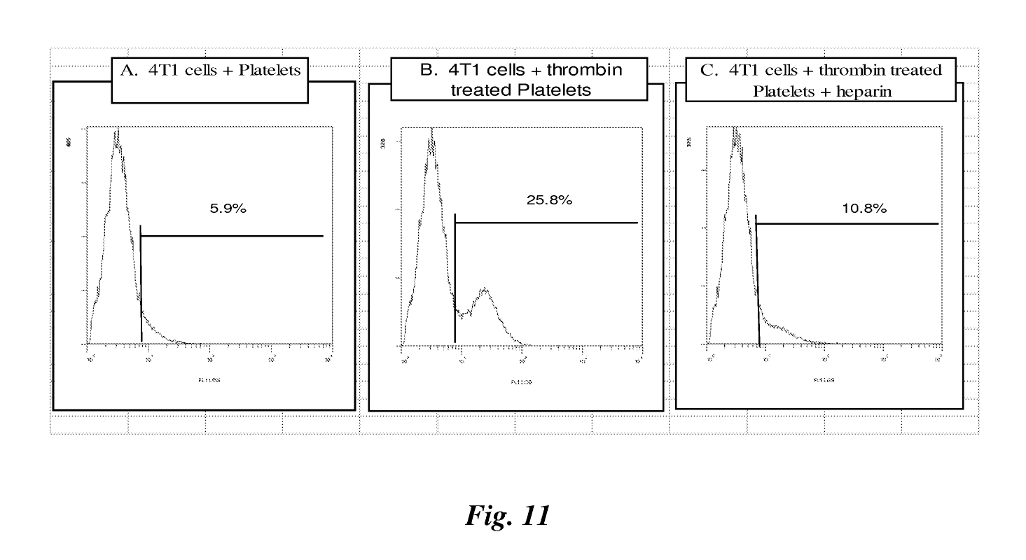

To examine if heparin can block the interaction of mouse platelets with tumor cells, 4T1 cells were mixed with Calcein-AM-labeled mouse platelets in the presence of mouse thrombin with or without heparin. Mouse thrombin was added to stimulate relocation of P-Selectin to platelet surface and tumor cells were then analyzed by flow cytometry for Calcein-AM staining, indicating platelet attachment. Thrombin treated platelets showed binding to tumor cells, which was reduced in the presence of heparin (FIG. 11). Blood was collected into sodium citrate (0.38% w/v) from naïve mice and platelets were isolated from plasma by centrifugation. Platelet were washed and labeled with 5 μM final Calcein-AM (Molecular Probes, Eugene, Oreg., USA) for 15 minutes at 37° C. Platelets were then washed and incubated with 1 U/ml thrombin (Haematologic Technologies Inc, Essex Junction, Vt.) at 37° C. for 10 minutes. Heparin (Baxter Healthcare Corp., Deerfield, Ill., 100 U/ml final concentration) was then added to the mixture of platelets and thrombin, and incubated with 4T1 cells in flow cytometry tubes, for 15 minutes at room temperature, then acquired and analyzed by flow cytometry.

FIG. 11A illustrates lack of binding of 4T1 cells incubated with untreated platelets. FIG. 11B illustrates binding of 4T1 cells to platelets which had been pre-treated with thrombin. FIG. 11C illustrates that heparin can inhibit binding of 4T1 cells to platelets which had been pre-treated with thrombin. The data illustrate that tumor cell-platelet interaction is P-Selectin mediated and can be blocked by heparin.

### Example 12

It has been shown that heparin administration at clinically relevant dose inhibited lung metastasis in experimental models, where tumor cells were delivered directly into the blood stream (8). However, in order to translate the results into clinical practice, such experimental evaluations should be performed in syngeneic spontaneous models. The murine mammary 4T1 cell line is a perfect model. In particular, sLe^{x}-Neg variant is an appropriate model as it does not express overlapping selectin reactive epitopes sLe^{x/a}.

BALB/c female mice (6-8 weeks old) were purchased from Harlan (Indianapolis, Ind.). Tumors were established as described earlier (9). Briefly, each mouse was inoculated subcutaneously in the abdominal mammary gland with 5×10⁴ 4T1 cells. To establish a functional correlation between P-selectin ligand expression of 4T1 cells and their metastatic ability in vivo, we injected mice with 100 units of heparin 30 minutes before tumor cell inoculation. Mice were sacrificed 26 days after tumor inoculation, lungs were harvested and metastatic cells were detected by clonogenic assay. We observed a complete absence of metastases in lung of majority of mice (six mice out of total of seven) injected with heparin (Table 1). All mice that were injected with PBS as control developed lung metastasis. Thus, blocking of P-selectin interaction with its ligand in vivo significantly prevented establishment of metastatic foci.

TABLE I
Number of mice detected positive for established lung
metastases in groups administered with heparin or PBS.
Total number of mice examined in each experiment is
given in the parenthesis.

| Treatment | Positive (total) |
|---|---|
| PBS | 6 (6) |
| Heparin | *1 (7) |

*P = 0.0047 as compared with PBS treated group by Fisher exact test.

Similarly, no mice were detected positive for lung metastases after treatment with Chondroitinase ABC.

TABLE II
Number of mice detected positive for established lung
metastases after chondroitinase ABC treatment of the
cancer cells.

| Treatment | Positive (total) |
|---|---|
| ChABC | *0 (5) |
| Non-treated | 5 (5) |

*P = 0.0079 as compared with group injected with non-treated cells by Fisher exact test.

### Example 13

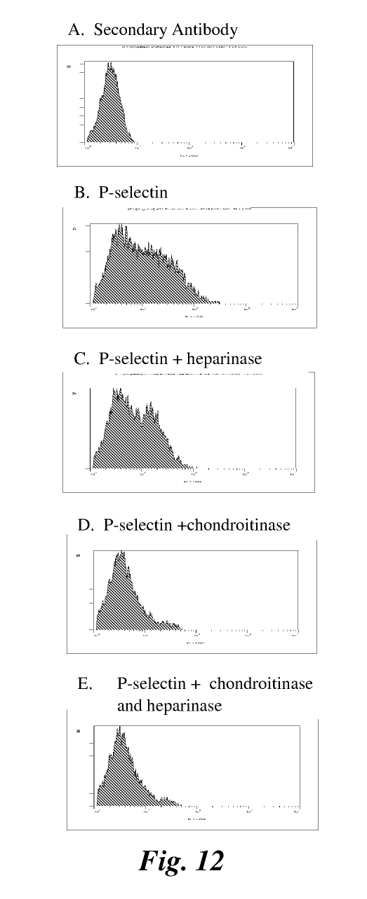

P-Selectin binds to CS PGs on the surface of human renal adenocarcinoma (10). To further explore the nature of P-Selectin ligands on the 4T1 tumor cell line, used heparinase and chondroitinase ABC were used separately in P-Selectin binding assays. The data indicate a major role for CS in P-Selectin binding to the 4T1 cells (FIG. 12). FIG. 12A illustrates 4T1 cells incubated with secondary antibody alone. FIG. 12B illustrates P-Selectin binding to 4T1 cells. FIG. 12C illustrates P-Selectin binding to 4T1 cells which had been pre-treated with heparinase. FIG. 12D illustrates P-Selectin binding to 4T1 cells which had been pre-treated with chondroitinase. FIG. 12E illustrates P-Selectin binding to 4T1 cells which had been pre-treated with both heparinase and chondroitinase.

### Example 14

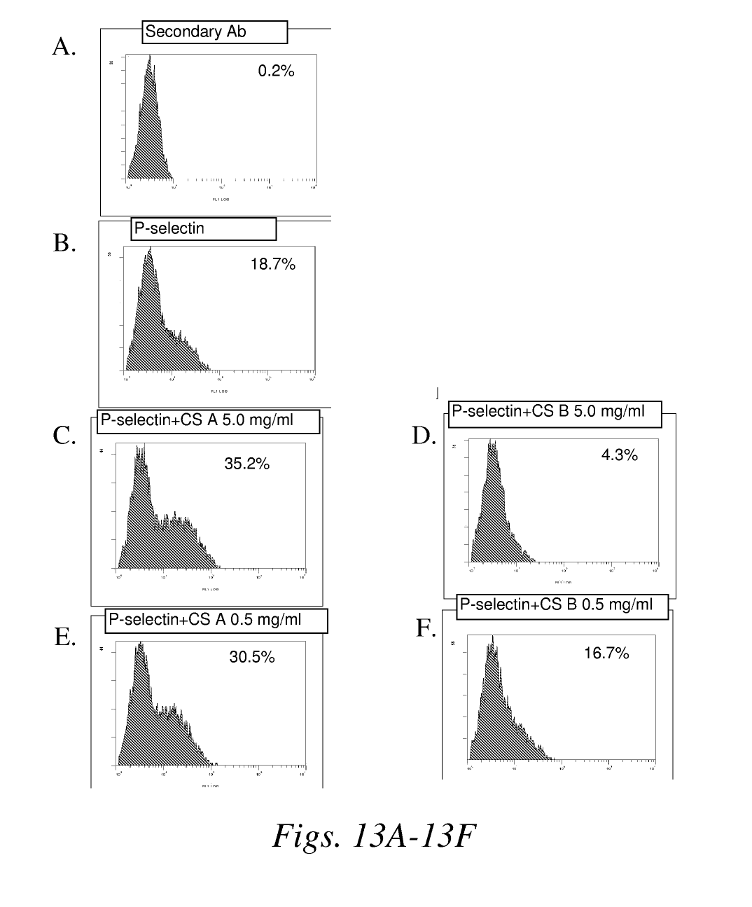

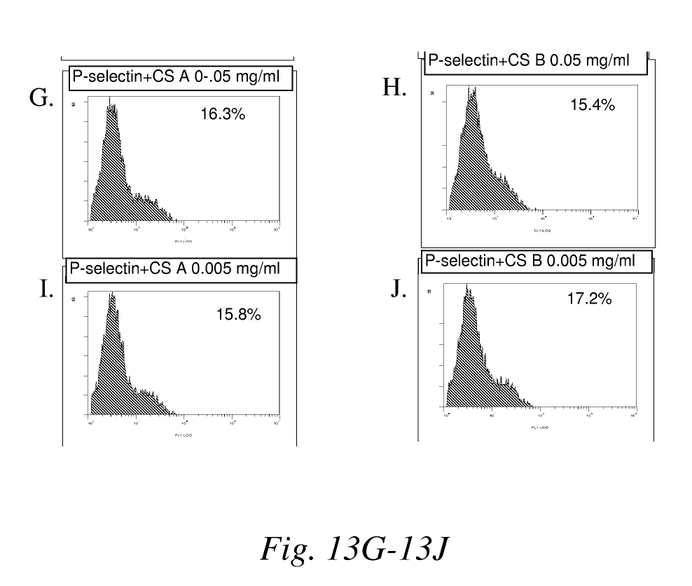

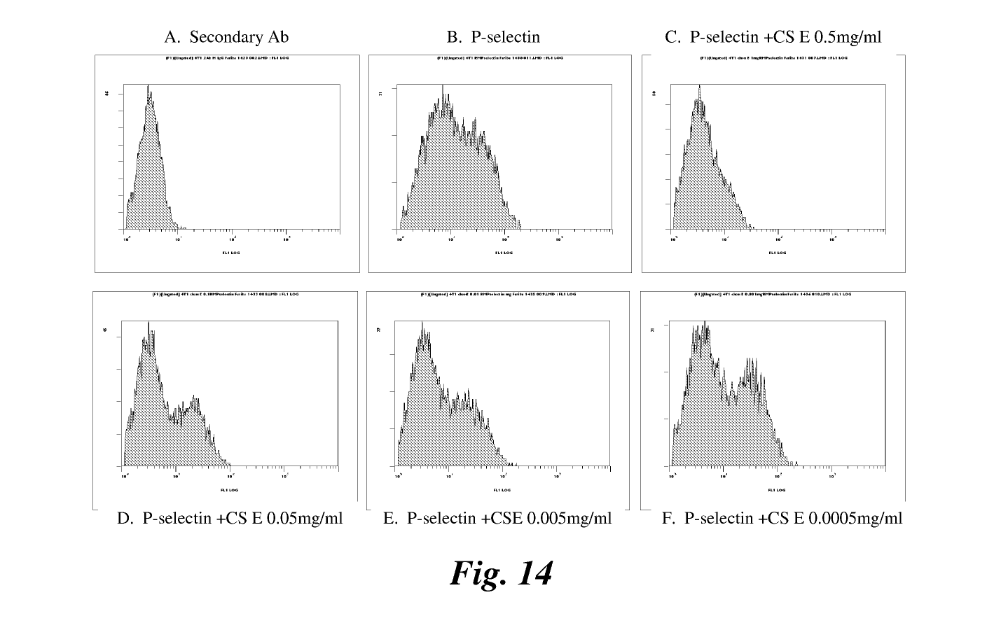

Chondroitin sulfates, including chondroitin sulfates A, B, C and E, block the interaction of P-Selectin to cancer cells. Binding of recombinant P-Selectin to cells was examined after treatment with heparinase and chondroitinase ABC or in the presence of various concentrations of heparin and chondroitin sulfate A, B, C, and E (Seikagaku America, Falmouth, Mass.). Among those tested, CS B (dermatan sulfate) and CS E inhibited P-Selectin binding to the cells (FIGS. 13 and 14). CS A and C (data not shown) showed minimal inhibitory effects. CS B showed inhibitory effects only at higher concentrations, while CS E was a more potent inhibitor with a complete inhibition at a concentration of 0.5 mg/ml. The effective dose of heparin, 120 units (FIG. 10), corresponds to 0.7 mg/ml heparin, which is close to the CS E blocking concentration of 0.5 mg/ml. FIG. 13A illustrates 4T1 cells incubated with secondary antibody alone. FIG. 13B illustrates P-Selectin binding to 4T1 cells. FIG. 13C illustrates P-Selectin binding to 4T1 cells when the P-Selectin had been pre-treated with 5.0 mg/ml chondroitin sulfate A. FIG. 13D illustrates P-Selectin binding to 4T1 cells when the P-Selectin had been pre-treated with 5.0 mg/ml chondroitin sulfate B. FIG. 13E illustrates P-Selectin binding to 4T1 cells when the P-Selectin had been pre-treated with 0.5 mg/ml chondroitin sulfate A. FIG. 13F illustrates P-Selectin binding to 4T1 cells when the P-Selectin had been pre-treated with 0.5 mg/ml chondroitin sulfate B. FIG. 13G illustrates P-Selectin binding to 4T1 cells when the P-Selectin had been pre-treated with 0.05 mg/ml chondroitin sulfate A. FIG. 13H illustrates P-Selectin binding to 4T1 cells when the P-Selectin had been pre-treated with 0.05 mg/ml chondroitin sulfate B. FIG. 13I illustrates P-Selectin binding to 4T1 cells when the P-Selectin had been pre-treated with 0.005 mg/ml chondroitin sulfate A. FIG. 13J illustrates P-Selectin binding to 4T1 cells when the P-Selectin had been pre-treated with 0.005 mg/ml chondroitin sulfate B.

### Example 15

FIG. 14A illustrates 4T1 cells incubated with secondary antibody alone. FIG. 14B illustrates P-Selectin binding to 4T1 cells. FIG. 14C illustrates P-Selectin binding to 4T1 cells when the P-Selectin had been pre-treated with 0.5 mg/ml chondroitin sulfate E. FIG. 14D illustrates P-Selectin binding to 4T1 cells when the P-Selectin had been pre-treated with 0.05 mg/ml chondroitin sulfate E. FIG. 14E illustrates P-Selectin binding to 4T1 cells when the P-Selectin had been pre-treated with 0.005 mg/ml chondroitin sulfate E. FIG. 14F illustrates P-Selectin binding to 4T1 cells when the P-Selectin had been pre-treated with 0.0005 mg/ml chondroitin sulfate E. These data indicate that chondroitin sulfate is the major P-selectin ligand on the surface of the 4T1 cells. Oversulfated CS E is able to effectively inhibit the interactions.

### Example 16

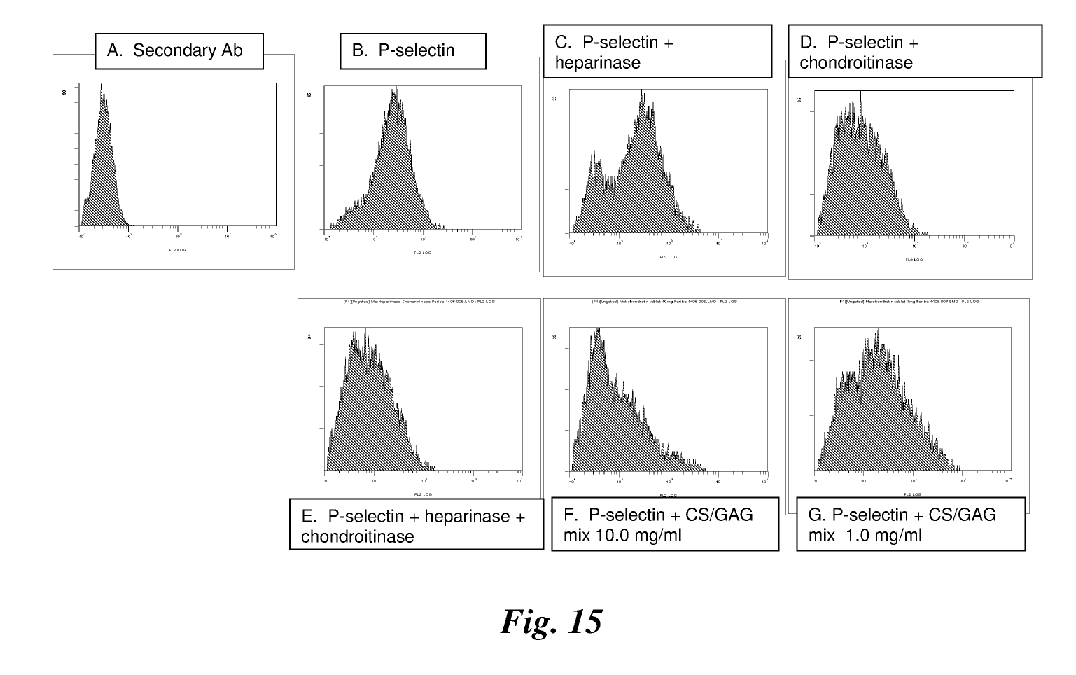

The bone-colonizing human breast cancer cell variant MDA-MET was tested for expression of P-Selectin ligands. P-Selectin reactivity with cells was decreased after chondroitinase treatment (FIG. 15). Heparinase or a mixture of heparinase and chondroitinase did not affect P-Selectin binding. This data suggests that CS PGs can also be a major P-Selectin ligand on the surface of human breast cancer cells. FIG. 15A illustrates MDA-MET cells incubated with secondary antibody alone. FIG. 15B illustrates P-Selectin binding to MDA-MET cells. FIG. 15C illustrates P-Selectin binding to MDA-MET cells when the cells had been pre-treated with heparinase. FIG. 15D illustrates P-Selectin binding to MDA-MET cells when the cells had been pre-treated with chondroitinase. FIG. 15E illustrates P-Selectin binding to MDA-MET cells when the cells had been pre-treated with both heparinase and chondroitinase. FIG. 15F illustrates P-Selectin binding to MDA-MET cells when the P-Selectin had been pre-treated with 10.0 mg/ml of a chondroitin sulfate and glycosaminoglycan mixture. FIG. 15G illustrates P-Selectin binding to MDA-MET cells when the P-Selectin had been pre-treated with 1.0 mg/ml of a chondroitin sulfate and glycosaminoglycan mixture.

Characterization of P- and E-selectin ligands is important for the assessment of metastatic risk and the development of possible ways of dealing with metastatic disease. A significant amount of P-Selectin binding is both Ca²⁺-independent and sialic acid-independent, confirming that sLe^{x} is not a P-Selectin ligand on 4T1 cells.

While sLe^{x/a} oligosaccharides are common ligands for both E- and P-Selectin, these two lectins do not correlate in their reactivity with the 4T1 cells. P-Selectin binds to the 4T1 cells strongly and the binding is not affected by sorting for sLe^{x} oligosaccharide by KM93 antibody or even by FTIII gene transfection. E-selectin binding can be predicted by reactivity of anti-sLe^{x} antibodies, indicating that E-selectin binding is predominately sLe^{x} dependent. However, P-Selectin binding did not correlate with either E-selectin or sLe^{x}-reactive antibodies, suggesting that much of the P-Selectin binding is not to sLe^{x} or other related oligosaccharides. There is a correlation between sLe^{x} reactive antibody binding and E-selectin binding to tumor cells but no such correlation to P-Selectin binding. The P-Selectin binding in 4T1 is dependent upon structures other than sLe^{x} or sLe^{a} ligands with increased sLe^{x} expression having almost no effect on P-Selectin binding.

E-selectin binding to the 4T1 cell line is restricted to sLe^{x} or closely related structures while P-Selectin binding can involve a varied group of compounds, including Ca²⁺-independent binding to non-Lewis structures. Characterization of P-Selectin binding to the 4T1 cells illustrates that this interaction is sulfur dependent and heparinase/chondroitinase sensitive. Further characterization of the 4T1 surface ligands clearly indicate that CS and CS glycosaminoglycans are the major P-Selectin ligands expressed on this cell line. CS B and CS E are able to inhibit the interaction.

The stable expression of P-Selectin ligands on 4T1 cells in vivo suggests that these ligands contribute to the metastatic behavior of this cell line. Cell surface P-Selectin ligands indeed contribute to binding of the 4T1 cells to platelets and HUVECs. Intact P-Selectin reactivity with heparan sulfate or CS may facilitate microemboli formation and adhesion to the endothelial cells, promoting tumor cell arrest in vasculature and extravasation.

Heparin is being used as anticoagulant treatment of venous thromboembolism in cancer patients, where it has been shown to improve patient survival by mechanisms not explained by anticoagulation (11). The present invention clearly demonstrates that Heparin inhibited P-Selectin binding to the 4T1 cells, and it blocked P-Selectin mediated adhesion of platelets to this tumor cell line. This data warranted in vivo testing of heparin for inhibition of metastasis in tumor bearing animals.

Inhibition of interaction between P-Selectin with its various ligands on tumor cells has an anti-metastatic therapeutic effect. Competition studies demonstrate that heparin and CS interaction may involve a region of the P-Selectin molecule very close to the lectin binding site for sLe^{x}. Heparin is capable of blocking P-Selectin binding to various tumor cells with various surface ligands, including sLe^{x} (12), sulfated glycolipids (13), heparan sulfate PGs (14, 15) and even CS PGs (10). In addition, the binding of a CS proteoglycan to P-Selectin was inhibited by sLe^{x}, which is in agreement with the notion that CS binding to the lectin domain of P-Selectin is similar to sLe^{x} binding (10). The present data suggest that highly sulfated CS types may be used to block P-Selectin binding to any of its ligands on tumor cells. Such broad specificity can be explained by recognition of a clustered epitope by P-Selectin (6). Targeting P-Selectin interaction with these ligands can be used for treatment of metastatic cancer. The current data support administration of CS as an alternative to treat metastatic disease.

As various changes could be made in the above methods and compositions without departing from the scope of the invention, it is intended that all matter contained in the above description be interpreted as illustrative and not in a limiting sense. Unless explicitly stated to recite activities that have been done (i.e., using the past tense), illustrations and examples are not intended to be a representation that given embodiments of this invention have, or have not, been performed.

All references cited in this specification are hereby incorporated by reference in their entirety. The discussion of the references herein is intended merely to summarize the assertions made by their authors and no admission is made that any reference constitutes prior art relevant to patentability. Applicant reserves the right to challenge the accuracy and pertinence of the cited references.

As various changes could be made in the above methods and compositions without departing from the scope of the invention, it is intended that all matter contained in the above description be interpreted as illustrative and not in a limiting sense. Unless explicitly stated to recite activities that have been done (i.e., using the past tense), illustrations and examples are not intended to be a representation that given embodiments of this invention have, or have not, been performed.

### REFERENCES

1. Fukushi, Y., Nudelman, E., Levery, S. B., Hakomori, S., and Rauvala, H. Novel fucolipids accumulating in human adenocarcinoma. III. A hybridoma antibody (FH6) defining a human cancer-associated difucoganglioside (VI3NeuAcV3III3Fuc2nLc6). J Biol Chem, 259: 10511-10517, 1984.
2. Fukushima, K., Hirota, M., Terasaki, P. I., Wakisaka, A., Togashi, H., Chia, D., Suyama, N., Fukushi, Y., Nudelman, E., and Hakomori, S. Characterization of sialosylated Lewisx as a new tumor-associated antigen. Cancer Res, 44: 5279-5285, 1984.
3. Hanai, N., Shitara, K., and Yoshida, H. Generation of monoclonal antibodies against human lung squamous cell carcinoma and adenocarcinoma using mice rendered tolerant to normal human lung. Cancer Res, 46: 4438-4443, 1986.
4. Hoff, S. D., Matsushita, Y., Ota, D. M., Cleary, K. R., Yamori, T., Hakomori, S., and Irimura, T. Increased expression of sialyl-dimeric LeX antigen in liver metastases of human colorectal carcinoma. Cancer Res, 49: 6883-6888, 1989.
5. Dohi, T., Nemoto, T., Ohta, S., Shitara, K., Hanai, N., Nudelman, E., Hakomori, S., and Oshima, M. Different binding properties of three monoclonal antibodies to sialyl Le(x) glycolipids in a gastric cancer cell line and normal stomach tissue. Anticancer Res, Vol. 13, pp. 1277-1282, 1993.
6. Koenig, A., Norgard-Sumnicht, K., Linhardt, R., and Varki, A. Differential interactions of heparin and heparan sulfate glycosaminoglycans with the selecting. Implications for the use of unfractionated and low molecular weight heparins as therapeutic agents. J Clin Invest, 101: 877-889, 1998.
7. Kawashima, H., Atarashi, K., Hirose, M., Hirose, J., Yamada, S., Sugahara, K., and Miyasaka, M. Oversulfated chondroitin/dermatan sulfates containing GlcAbeta1/IdoAalpha1-3GalNAc(4,6-O-disulfate) interact with L- and P-Selectin and chemokines. J Biol Chem, 277: 12921-12930, 2002.
8. Stevenson, J. L., Choi, S. H., and Varki, A. Differential metastasis inhibition by clinically relevant levels of heparins--correlation with selectin inhibition, not antithrombotic activity. Clin Cancer Res, 11: 7003-7011, 2005.
9. Monzavi-Karbassi, B., Artaud, C., Jousheghany, F., Hennings, L., Carcel-Trullols, J., Shaaf, S., Korourian, S., and Kieber-Emmons, T. Reduction of Spontaneous Metastases through Induction of Carbohydrate Cross-Reactive Apoptotic Antibodies. J Immunol, 174: 7057-7065, 2005.
10. Kawashima, H., Hirose, M., Hirose, J., Nagakubo, D., Plaas, A. H., and Miyasaka, M. Binding of a large chondroitin sulfate/dermatan sulfate proteoglycan, versican, to L-selectin, P-Selectin, and CD44. J Biol Chem, 275: 35448-35456, 2000.
11. Cosgrove, R. H., Zacharski, L. R., Racine, E., and Andersen, J. C. Improved cancer mortality with low-molecular-weight heparin treatment: a review of the evidence. Semin Thromb Hemost, 28: 79-87, 2002.
12. Nelson, R. M., Cecconi, O., Roberts, W. G., Aruffo, A., Linhardt, R. J., and Bevilacqua, M. P. Heparin oligosaccharides bind L- and P-Selectin and inhibit acute inflammation. Blood, 82: 3253-3258, 1993.
13. Borsig, L., Wong, R., Hynes, R. O., Varki, N. M., and Varki, A. Synergistic effects of L- and P-Selectin in facilitating tumor metastasis can involve non-mucin ligands and implicate leukocytes as enhancers of metastasis. Proc Natl Acad Sci USA, 99: 2193-2198, 2002.
14. Wei, M., Tai, G., Gao, Y., Li, N., Huang, B., Zhou, Y., Hao, S., and Zeng, X. Modified heparin inhibits P-Selectin-mediated cell adhesion of human colon carcinoma cells to immobilized platelets under dynamic flow conditions. J Biol Chem, 279: 29202-29210, 2004.
15. Ma, Y. Q. and Geng, J. G. Heparan sulfate-like proteoglycans mediate adhesion of human malignant melanoma A375 cells to P-Selectin under flow. J Immunol, 165: 558-565, 2000.

What is claimed is:

1. A composition for inhibiting metastasis of a cancer cell comprising a chondroitin sulfate ligand.

2. The composition of claim 1 wherein said chondroitin sulfate ligand comprises an adhesion molecule.

3. The composition of claim 2 wherein said adhesion molecule is soluble P-Selectin.

4. The composition of claim 1 wherein said chondroitin sulfate ligand blocks binding of P-Selectin on the surface of a cell.

5. The composition of claim 4 wherein said cell is a platelet cell or an endothelial cell.

6. The composition of claim 4 wherein said chondroitin sulfate ligand comprises an antibody.

7. A composition for inhibiting metastasis comprising a P-Selectin ligand.

8. The composition of claim 7 wherein said P-Selectin ligand comprises chondroitin sulfate.

9. The composition of claim 8 wherein said chondroitin sulfate comprises a chondroitin sulfate proteoglycan.

10. The composition of claim 8 wherein said chondroitin sulfate is selected from the group consisting of chondroitin sulfate A, chondroitin sulfate B, chondroitin sulfate C, chondroitin sulfate D, and chondroitin sulfate E.

11. A composition for inhibiting metastasis comprising an inhibitor of synthesis of chondroitin sulfate.

12. The composition of claim 11 wherein said inhibitor inhibits an enzyme selected from the group consisting of chondroitin synthase, chondroitin N-acetylgalactosaminyltransferase (Chondroitin GalNAcT), chondroitin-glucuronate C5-epimerase, chondroitin 4-O-sulfotransferase-1 (C4ST1), chondroitin 4-O-sulfotransferase-2 (C4ST2), chondroitin 4-O-sulfotransferase-3 (C4ST3), dermatan 4-O-sulfotransferase-1 (D4ST1), chondroitin 6-O-sulfotransferase (C6ST), chondroitin 6-O-sulfotransferase-2 (C6ST2), chondroitin 4-sulfate 6-O-sulfotransferase (GalNAc4S-6ST) and galactosaminyl uronyl 2-O sulfotransferase (CS/DS2ST).

13. A method of inhibiting metastasis comprising blocking the interaction of a first cell comprising chondroitin sulfate with a second cell by contacting said first cell with a chondroitin sulfate ligand.

14. The method of claim 13 wherein said first cell is a cancer cell.

15. The method of claim 14 wherein said cancer cell is selected from the group consisting of breast cancer, colon cancer, lung cancer, malignant melanoma, gastric cancer, tongue squamous cancer, myeloma and neuroblastoma.

16. The method of claim 13 wherein said second cell is a platelet cell or an endothelial cell.

17. The method of claim 13 wherein said chondroitin sulfate ligand is soluble P-Selectin.

18. The method of claim 13 wherein said chondroitin sulfate ligand is an antibody that binds to chondroitin sulfate.

19. A method of inhibiting metastasis comprising blocking the interaction of a first cell comprising chondroitin sulfate with a second cell comprising P-Selectin by contacting said second cell with a P-Selectin ligand.

20. The method of claim 19 wherein said first cell is a cancer cell.

21. The method of claim 20 wherein said cancer cell is selected from the group consisting of breast cancer, colon cancer, lung cancer, malignant melanoma, gastric cancer, tongue squamous cancer, myeloma and neuroblastoma.

22. The method of claim 19 wherein said second cell is a platelet cell or an endothelial cell.

23. The method of claim 19 wherein said P-Selectin ligand is selected from the group consisting of chondroitin sulfate A, chondroitin sulfate B, chondroitin sulfate C, chondroitin sulfate D, and chondroitin sulfate E.

24. The method of claim 19 wherein said P-Selectin ligand comprises an antibody that binds to P-Selectin.

25. A method of inhibiting metastasis comprising contacting a cancer cell with a chondroitin sulfate synthesis inhibitor.

26. The method of claim 25 wherein said inhibitor inhibits an enzyme selected from the group consisting of chondroitin synthase, chondroitin N-acetylgalactosaminyltransferase (Chondroitin GalNAcT), chondroitin-glucuronate C5-epimerase, chondroitin 4-O-sulfotransferase-1 (C4ST1), chondroitin 4-O-sulfotransferase-2 (C4ST2), chondroitin 4-O-sulfotransferase-3 (C4ST3), dermatan 4-O-sulfotransferase-1 (D4ST1), chondroitin 6-O-sulfotransferase (C6ST), chondroitin 6-O-sulfotransferase-2 (C6ST2), chondroitin 4-sulfate 6-O-sulfotransferase (GalNAc4S-6ST) and galactosaminyl uronyl 2-O sulfotransferase (CS/DS2ST).

27. The method of claim 25 wherein said metastasis is selected from the group consisting of breast cancer metastasis, colon cancer metastasis, lung cancer metastasis, malignant melanoma metastasis, gastric cancer metastasis, tongue squamous cancer metastasis, myeloma metastasis and neuroblastoma metastasis.
